(19) United States
(12) Patent Application Publication
(10) Pub. No.: US 2004/0172019 A1
(43) Pub. Date: Sep. 2, 2004

(54) Reinforcers for artificial disc replacement methods and apparatus

(76) Inventor: Bret A. Ferree, Cincinnati, OH (US)

Correspondence Address: Gifford, Krass, Groh, Sprinkle,, Anderson & Citkowski, PC, Suite 400, 280 N. Old Woodward Ave., Birmingham, MI, 48009, US

(21) Appl. No.: 10/191,639

(22) Filed: Jul. 9, 2002

Related U.S. Application Data
(63) Continuation-in-part of application No. 09/415,382, filed on Oct. 8, 1999, now Pat. No. 6,419,704.
(63) Continuation-in-part of application No. 09/580,231, filed on May 26, 2000, now Pat. No. 6,494,883.

(51) Int. Cl. A61B 17/56

(52) U.S. Cl. 606/61

(57) ABSTRACT

An improved reinforced intradiscal plug includes a mid section having opposing ends, and a rubber, polymeric or elastomeric mid section. The mid section is preferably bounded by a pair of rigid end plates, each positioned against a respective end of the mid section. The mid section in combination with the end plates may assume a generally cylindrical, threaded configuration permitting a screw-in installation. Alternatively, the mid section in combination with the end plates assumes a configuration suitable to an impacted installation. At least the mid section may include seriating or protrusions to hold the plug in position once installed. The end plates are at least partially metallic, with at least one link member passing through the mid section so as to interconnect the opposing end sections. One of the end plates may include a threaded bore, with one end of the link member being threaded to receive the threaded bore of that end plate. According to a further alternative embodiment, the link member, a fastener, and/or the end plate(s) may be advanced, causing the rubber, polymeric or elastomeric section to bulge for a tighter fit within the intradiscal space.

### REFERENCE TO RELATED APPLICATIONS

This application is a continuation-in-part of U.S. patent application Ser. No. 09/415,382, filed Oct. 8, 1999, and Ser. No. 09/580,231, filed May 26, 2000, the entire content of both applications being incorporated herein by reference.

### FIELD OF THE INVENTION

The present invention relates generally to orthopedic surgery and, in particular, to reinforcers for artificial disc replacements

### BACKGROUND OF THE INVENTION

With respect to spinal surgery wherein one or more vertebrae are fused, the use of bone dowels have certain advantages over metal cages. First, allograft bone readily fuses to the vertebrae. Second, it is often impossible to determine if metal bone-filled cages have fused to adjacent vertebrae, because the metal obstructs x-ray imaging of the bone within the metal cages as well as the cage vertebra junction. Third, bone dowels have a modulus of elasticity closer to that vertebrae. Consequently, bone dowels stress shield less than metal cages.

Bone dowels have certain disadvantages when compared to metal cages, however. Allograft bone incorporates into host bone through a process known as “creeping substitution.” Host blood vessels grow into the allograft bone in the first stage of this process. Bone removing cells known as osteoclasts then invade the allograft bone. After sufficient bone is removed by the osteoclasts, bone building cells known as osteoblasts lay down new host bone on the allograft bone.

This remodeling process may go on for years. As would be expected, the allograft is weakened by the channels formed by the blood vessels, as well as the bone removal by the osteoclasts. Although the allograft regains its strength once sufficient new bone is formed, allograft bone dowels are at risk of fracture during the period of time that they are weakened. Allograft bone dowel fracture is well known to those skilled in the art of spinal surgery. Bone dowels are also weaker than metal cages, even before they undergo creeping substitution. Consequently, bone dowels can fracture during surgical placement. Fractured dowels can be difficult to remove, and may lead to failure of a fusion to occur. The properties of bone also do not allow certain shapes or machining.

### SUMMARY OF THE INVENTION

This invention is directed to an improved, reinforced intradiscal plug wherein the preferred embodiment includes a mid section having opposing ends, and a rubber, polymeric or elastomeric mid section. The mid section is preferably bounded by a pair of rigid end plates, each positioned against a respective end of the mid section.

In the preferred embodiment, the mid section in combination with the end plates assumes a generally cylindrical, threaded configuration permitting a screw-in installation. Alternatively, the mid section in combination with the end plates assumes a configuration suitable to an impacted installation. In certain embodiments, at least the mid section may include seriating or protrusions to hold the plug in position once installed.

The end plates are at least partially metallic, with at least one link member passing through the mid section so as to interconnect the opposing end sections. One of the end plates may include a threaded bore, with one end of the link member being threaded to receive the threaded bore of that end plate. According to a further alternative embodiment, the link member or a fastener on the link member may be advanced, causing the rubber, polymeric or elastomeric section to bulge.

### BRIEF DESCRIPTION OF THE DRAWINGS

FIG. 1 is an isometric view of a prior-art threaded cylindrical metal cage;

FIG. 2 is an isometric view of a prior-art cylindrical, threaded bone dowel;

FIG. 3 is a perspective, exploded view drawing of certain components associated with one embodiment of the invention;

FIG. 4 is an isometric view of the embodiment of FIG. 3 in an assembled form;

FIG. 5A is an exploded-view drawing depicting an alternative embodiment of the invention having threaded end sections;

FIG. 5B is a drawing of a completed assembly according to the invention of FIG. 5A;

FIG. 6A is an end component associated with an alternative embodiment of the invention;

FIG. 6B is a progression of the embodiment of the invention introduced to with respect to FIG. 6A;

FIG. 6C is a drawing of a completed assembly according to the invention of FIGS. 6A and 6B;

FIG. 7A begins a sequence of drawings showing how multiple bone dowels and plates may be stacked to provide a further alternative embodiment of the invention;

FIG. 7B shows the stacking of a first bone dowel and the receipt of a first end plate;

FIG. 7C shows the receipt of a second bone dowel;

FIG. 7D shows the receipt of an end plate;

FIG. 7E is a drawing which illustrates a completed stacked structure according to a further alternative embodiment of the invention;

FIG. 8A is a side-view drawing depicting an alternative method according to the invention wherein a bone reinforcer is assembled within an intervertebral disc space as opposed to being inserted as a finished component;

FIG. 8B is a drawing which shows a progression which began with reference to FIG. 8A;

FIG. 8C continues the progression of FIGS. 8A and 8B, with the addition of an end-cap;

FIG. 8D shows the assembled reinforcer using the steps of FIGS. 8A-8C;

FIG. 8E is a side-view drawing which shows how multiple disc spacers may be added during an in situ assembly;

FIG. 9A is a drawing of an extension post used in assembling a bone-reinforcer within a disc space;

FIG. 9B is a drawing which shows how an end-cap may be fastened to a central member such as a circular rod;

FIG. 9C is a drawing of an alternative embodiment wherein a reverse thread is used as opposed to a separate fastener with respect to an end plate;

FIG. 10A is a side-view drawing of an alternative impacted embodiment assembled within a disc space;

FIG. 10B is a side-view drawing of a completed structure according to the practice of FIG. 10A, showing, in particular, the use of a retaining clip;

FIG. 11 is a side-view drawing of an alternative embodiment of the invention wherein a central member includes projections which holds loose bone graft material in position;

FIG. 12 is a side-view drawing of yet a further different alternative embodiment of the invention, wherein spikes of different length are used for the purpose of holding bone graft and for holding the completed structure within place within an intervertebral space;

FIG. 13A is a drawing which shows yet a further, different alternative embodiment of the invention, wherein a central member used to connect end plates is itself open through the use of multiple structural members;

FIG. 13B is a drawing which shows the device of FIG. 13A with bone-graft material packed in and around the central member;

FIG. 14A illustrates a way in which multiple struts may be used, each terminating at both end plates;

FIG. 14B illustrates the use of multiple struts with one or more intermediate spacers;

FIG. 15A is a drawing which shows the use of multiple structural members between end sections in an impacted, as opposed to threaded, embodiment;

FIG. 15B is a drawing of an impacted embodiment wherein multiple struts are used lengthwise between the end sections;

FIG. 15C is a drawing which shows an impacted embodiment of the invention having an intermediate member, again interconnected with multiple struts;

FIG. 16A is a side view of the device according to the invention including a mid section composed of rubber or other polymeric/elastomeric material;

FIG. 16B is an end-view of the device of FIG. 16A;

FIG. 17A is a side-view drawing of the reinforcer of the type shown in FIGS. 16A and 16B, disposed between adjacent vertebra;

FIG. 17B is an anterior view showing the two cylindrical reinforcers in place;

FIG. 18A is a drawing of an alternative embodiment of the invention from a perspective view;

FIG. 18B shows the two devices of FIG. 18A cooperating with one another;

FIG. 19A illustrates yet a further embodiment of the invention, including an impacted rectangular shape with surface teeth or roughenings to prevent migration of the device;

FIG. 19B is an end-view of two devices of the type shown in FIG. 19A;

FIG. 20 shows an alternative view of the artificial disc device made of a stiffer material such as polyethylene;

FIG. 21A illustrates an embodiment of the invention in a pre-compressed/expanded condition;

FIG. 21B shows the device of FIG. 21A with a nut moved along a reinforcing shaft;

FIG. 21C is a cross-section of the devices of FIGS. 21A and 21B;

FIG. 21D is an end-view of the devices of FIGS. 21A-21C;

FIG. 22 shows the way in which instrumentation may be used to expand an enforcer according to the invention;

FIG. 23A shows how, after forming a hole in the annulus fibrosis and removing perhaps a portion of the nucleus pulposis, a tool is used to tighten onto a rod;

FIG. 23B shows how a second expandable member or cap may be added to the present invention; and

FIG. 23C shows the final appearance of the structure, having performed the steps of FIGS. 23A and 23B.

### DETAILED DESCRIPTION OF THE INVENTION

FIGS. 1 and 2 are perspective-view drawings of existing interbody fusion devices, with FIG. 1 being rendered in the form of a metal cage, and FIG. 2 being implemented in the form of a bone dowel. In both cases, the bodies 102 and 202 include respective apertures 104 and 204 to receive bone graft material to enhance fusing. The use of an all-metal component has its disadvantages, as does the use of an all-bone component as discussed above with respect to the background of the invention, such that those sufficiencies will not be repeated here. Broadly, the instant invention combines the judicious use of metal and bone components in reinforcers of this type, to gain the advantages of using both materials while avoiding the disadvantages.

FIG. 3 is an exploded view drawing of certain components of the invention, which are preferably fabricated from a biocompatible metal, metallic component or alternative material sufficient to impart strength to the finished article. The components in this case include an end plate 306 having attached thereto a rod of smaller diameter 308, terminating in a threaded end 310, preferably further including a recess 312 to receive a tool such as an allen wrench, screwdriver, and so forth, to be used for stabilization and/or tightening.

Onto the threaded end 310, there is received a second outer plate 320 having threads 322 which mate with the threads 310. These are preferably reverse threads, so that they tighten rather than loosen when the device is installed. Although the rod 308 is shown preferably permanently connected to the end plate 306, it will be appreciated that a threaded, preferably reverse-threaded connection may be provided there as well.

FIG. 4 is a drawing which shows how the components of FIG. 3 are assembled to produce a finished reinforcer according to the invention. Broadly, the end plates 306 and 320 are spaced apart when assembled to provide a spacer therebetween to receive a section of bone grating material 400 preferably including one or more apertures 402 to receive bone graft material sufficient to enhance fusion. Note that the threads 406 on the device overall are forwardly oriented, such that, by placing an appropriate tool into the aperture 322, the plug may be rotated into place without the threaded connection(s) of the end plates becoming loose.

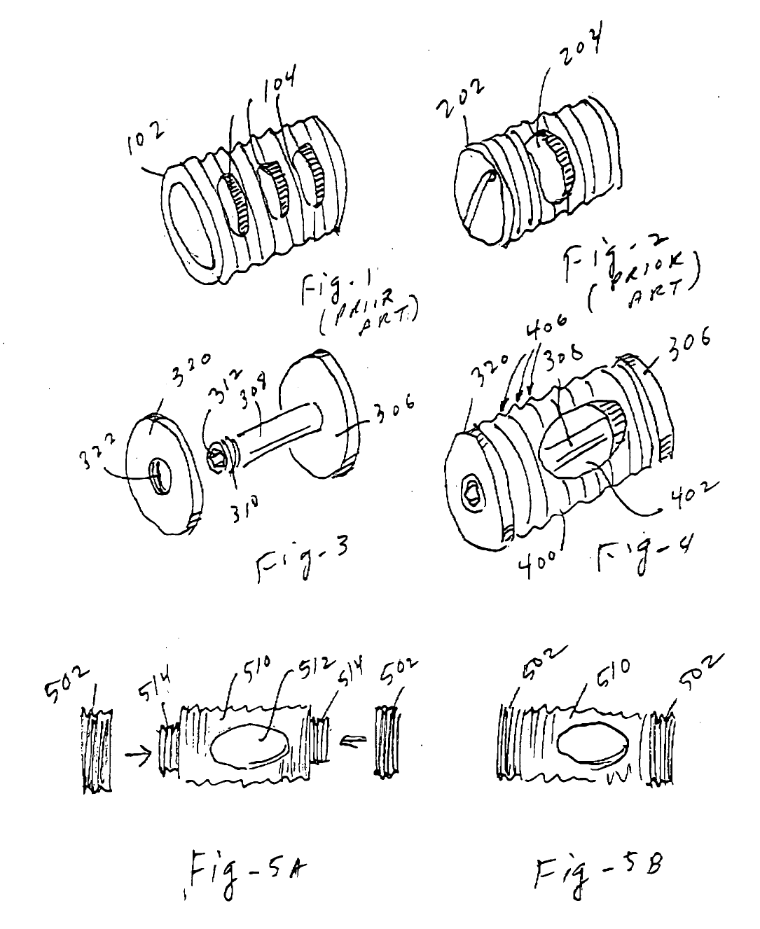

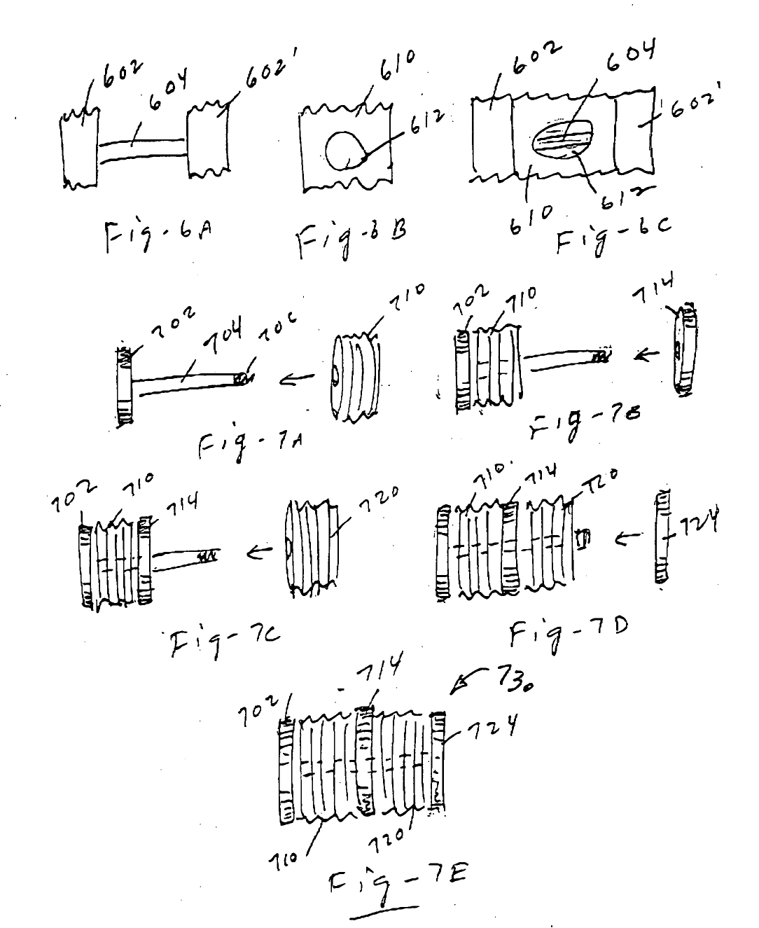

FIGS. 5A and 5B represent an alternative embodiment of the invention, wherein rigid discs 502, preferably of metal, having inner threads enabling them to be rotated onto a dowel 510 of bone material having threaded end sections 514 and one or more apertures 512. The end plates 502 are rotated onto the end sections 514 of the dowel 510, preferably until they become flush with the body of the dowel, as shown in FIG. 5B. Note that since the end plates are preferably tightened against the ends of the dowel, forward or reverse threads may alternatively be used for such purpose.

FIGS. 6A-6C illustrate a different embodiment of the invention, which may be used to produce finished articles of various shapes, including cylindrical, rectangular, trapezoidal, and other geometries. As shown in FIG. 6A, the structure includes two end pieces 602 and 602′, these being attached with a member 604 so that they are spaced apart from one another by an appropriate distance to receive the bone section 610 having one or more apertures 612 illustrated in FIG. 6B. In this case, it is noted that, as opposed to a helical thread disposed on the outer body of the device, teeth are provided on one or more opposing surfaces, such that the device is tapped into place as opposed to being rotatably inserted, thereby enabling the shape to non-circular in cross-section. FIG. 6C shows the completed structure, with the insert of FIG. 6B being installed onto the supports shown in FIG. 6A.

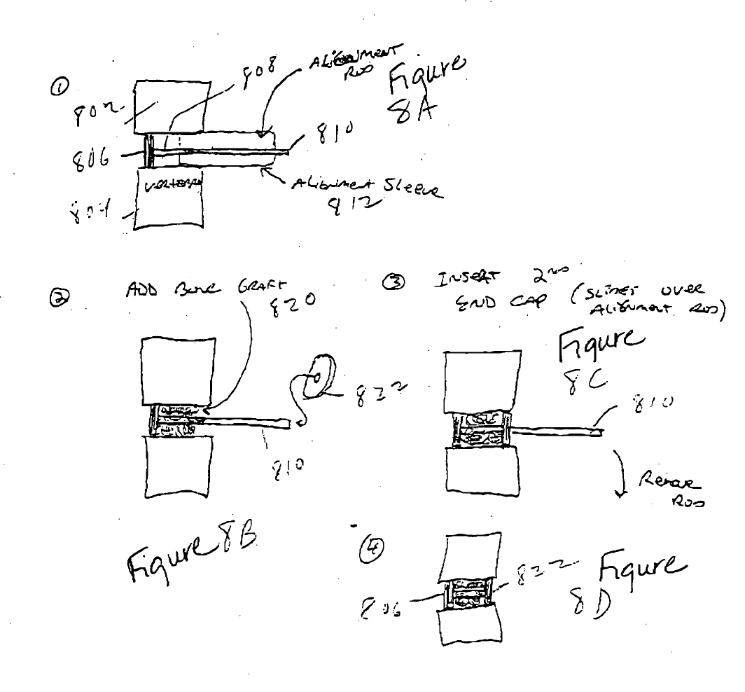

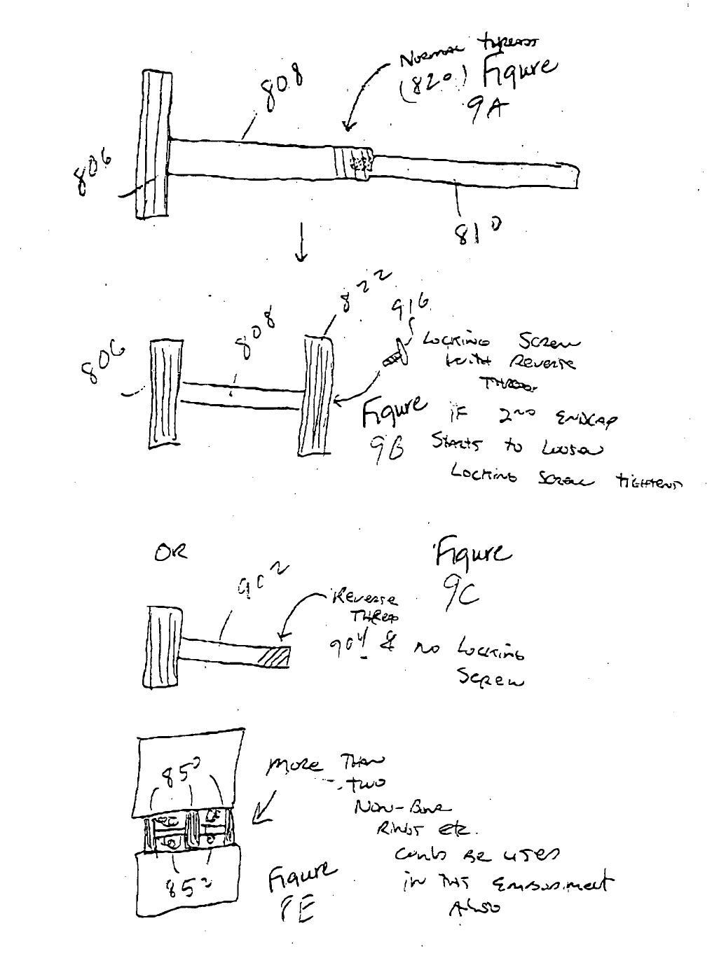

Although the embodiments so far described generally illustrate two end plates separated from one another having a bone insert therebetween, the invention is not limited as to the number of plates or spacers, and may use intermediate discs or rings along the body of the device. FIG. 7E is a drawing which shows such a finished article generally at 730, having at least one non-bone spacer along the length of the device.

FIGS. 7A-7D show how such a device would preferably be assembled, namely beginning with a first plate 702 coupled to a rod 704 having a threaded end 706, a first section of bone material 710 would be journaled onto the rod 704, as shown in FIG. 7A. A second non-bone ring 714 would then be added, as shown in FIG. 7B, followed by a second piece of bone 720, as shown in FIG. 7C. A final end plate 724, having an internal threaded bore which cooperates with the threaded end 706 of the rod 704 would then be added, as shown in FIG. 7B, to achieve the finished structure shown in FIG. 7E, generally at 730.

It will be appreciated by one of skill in the art, that more than two or three non-bone spacers may be used in any of the embodiments shown herein, and that autograft or all graft bone may be used, that is, bone from the same patient or a different individual. For that matter, synthetic bone material may be used as opposed to naturally occurring bone and, in addition to threaded connections between the various components, alternative assembly techniques such as compression or force-fit interfaces may be used.

Although the embodiments described thus far reside in completed reinforcers which are inserted into an intervertebral disc space, the invention is not limited to prefabricated structures, but, in fact, devices according to the invention may be assembled progressively within the disc space. FIGS. 8A-8D illustrate such a sequence of assembly, with FIG. 8E being used to show that multiple spacer plates in addition to the end plates may also be assembled in situ. In these figures, a bone reinforcer is being assembled in the disc space between upper and lower vertebrae 802 and 804, respectively.

The installation procedure may be carried out from an anterior or posterior approach. Assuming the latter, an anterior end plate 806 attached to a distal rod portion 808 is first installed, by pushing the end piece 806 into position using a detachable extender rod 810. To ensure that insertion progresses in a well-defined and controlled manner, an alignment sleeve 812 may temporarily be used as a guide. Having placed the anterior end piece, bone graft 820 is packed into the space around the rod 808, and a posterior end piece 822 is installed onto the assembly over extension 810. FIG. 8C shows the posterior end plate in position, at which time the extension piece 810 is removed, as shown in FIG. 8D, leaving only the bone graft material surrounding a central member connecting the two end plates 806 and 822.

As with other embodiments described herein, the invention is not limited to the use of rigid end pieces, but rather, intermediate discs or rigid elements may be used, including embodiments wherein the device is assembled within the disc space. Once such configuration is shown in FIG. 8E, wherein multiple plates 850 are used, with bone graft 852 being progressively added as each plate is installed.

FIGS. 9A-9C illustrate ways in which the extension member 810 may be removed, and the posterior end plate installed. FIG. 9A shows the anterior end plate 806, preferably rigidly attached to the central member 808, with extender 810 being attached thereto, along with the addition of normal or forward-oriented threads 820 located at the posterior terminating end of the member 808. Having removed the extender 810, the posterior end plate 822 may be installed through the use of a locking screw 916 having reversed threads from that of 820, to ensure that the last to install end plate does not become loose when the locking screw 916 is tightened down. As an alternative, the threads 820 may be reverse-oriented, as shown in FIG. 9C, in which case the end piece may be screwed on without the need for an addition locking screw or other mechanism.

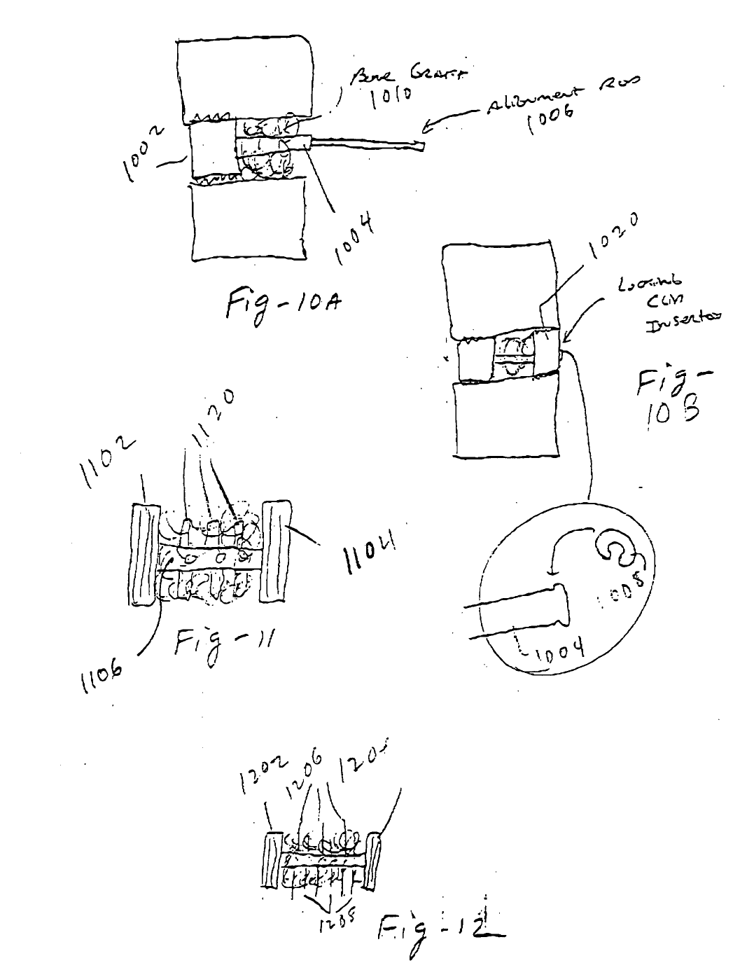

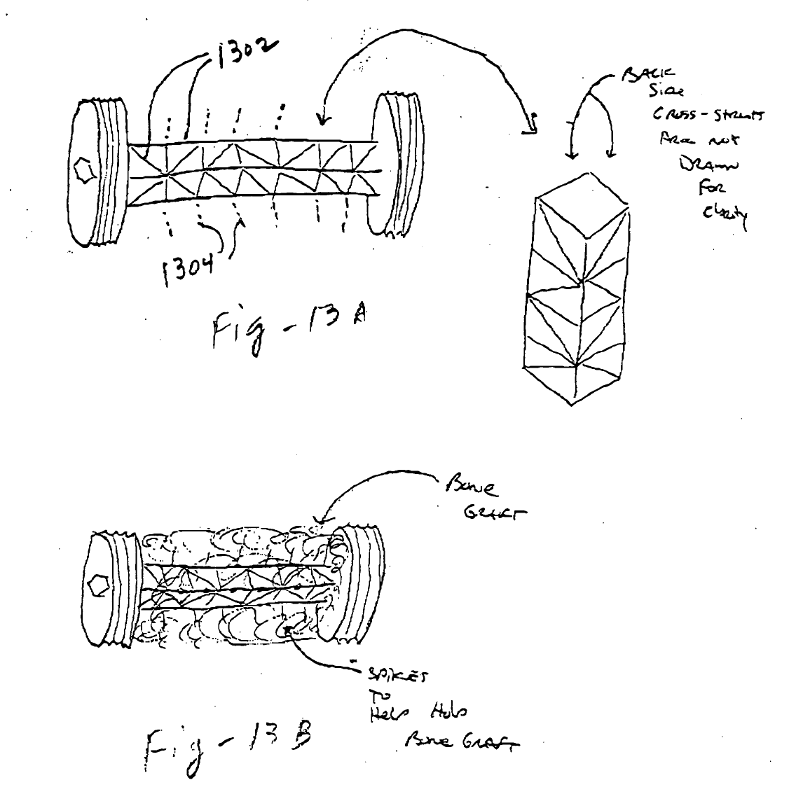

FIG. 10A illustrates a different alternative embodiment of the invention, wherein, as opposed to relatively thin end pieces, impactor plugs such as 1002 and 1020 are instead utilized. FIG. 10A illustrates the initial steps associated with the introduction of this assembly, with the anterior plug 1002 being forced into place, and bone graft material being added around the central member 1004. Again, an alignment rod 1006 is preferably temporarily used for placement. As shown in FIG. 10B, the second end plug 1020 is impacted into place over the central member 1004, at which time the alignment rod is removed. Since, in this embodiment, spiked or otherwise roughened superior and anterior surfaces are used on the plugs 1002 and 1020, a simplified retainer clip such as 1008 may be received by a corresponding groove in the central member 1004, as shown in the enlarged view. Although the plugs 1002 and 1020 may be circular in cross-section, in this particular embodiment they are preferably rectangular in cross-section, allowing a larger surface area for superior and inferior end plate engagement.

FIGS. 11 and 12 illustrate other alternative embodiments of the invention, including the use of spikes or rod emanating from the central member between the end pieces or intermediate pieces. Such a configuration may be used with prefabricated components according to the invention or, alternatively, assembled in place between the disc space. In FIG. 11, two end plates 1102 and 1104 are used, though others may be added lengthwise along the central member 1106, but from the member 1106, protrusions 1120 are provided. Depending upon their composition, and strength, the protrusions 1120 may provide additional support along the length of the reinforcer, but in addition, the protrusions 1120 act to hold the bone graft material in place, thereby further enhancing fusion.

Although the protrusions such as 1120 shown in FIG. 11 may be uniform in length as measured from the central member connecting the end plates or intermediate pieces, as shown in FIG. 12, protrusions could also be used which are collectively wider in cross-section than the intervertebral space, such that, during insertion, they are bent down and engage with the end plates, thereby preventing the completed structure from backing out while, at the same time, holding the bone graft material in position. These longer protrusions such as 1206, may be used in combination with shorter protrusions 1208, with the shorter protrusions being specifically intended to hold the bone graft material in place, while the longer protrusions act as barbs to hold the overall structure in position.

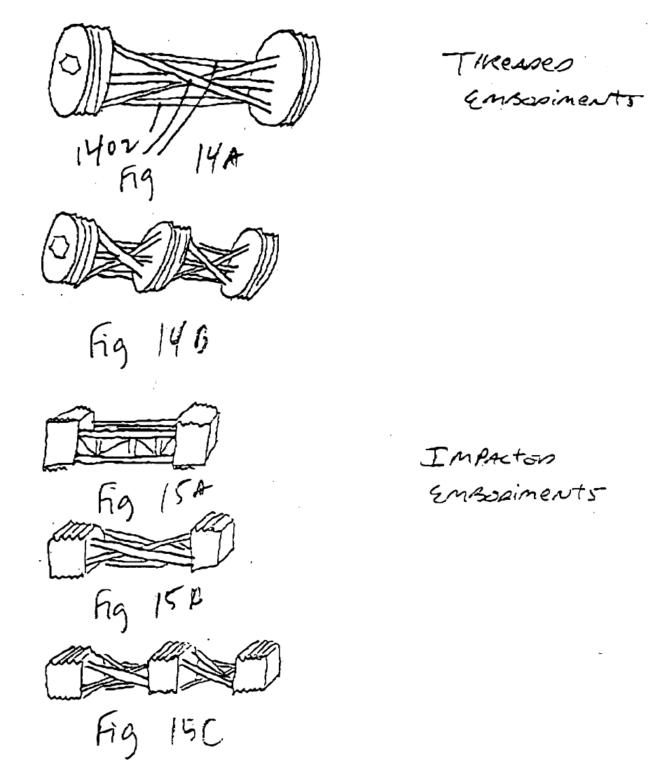

As opposed to a solid central member connecting end plates, multiple structural members 1302 may be used for an open core, as shown in the threaded embodiment of FIG. 13A. In conjunction with these cross-members, which may be arranged much like those found on a radio tower, optional spikes 1304 may be used to help hold bone graft into position, as shown in FIG. 13B. As a further alternative arrangement, the multiple struts may go from end section to end section, as shown in the threaded embodiment of FIG. 14A.

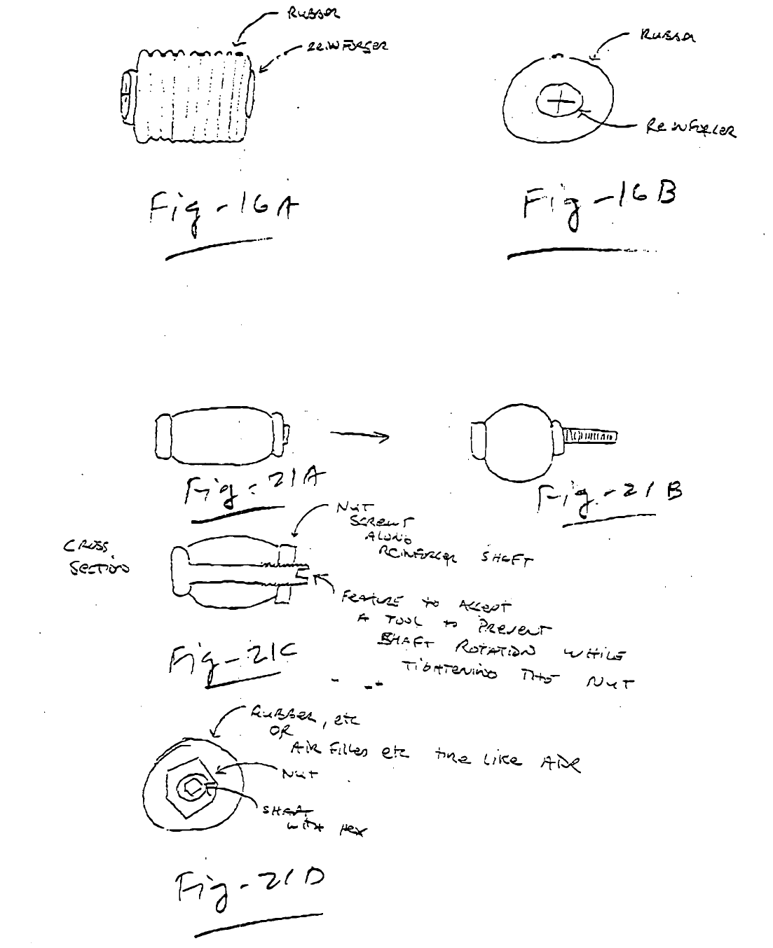

Intermediate spacers may be used in all of these embodiments, including those which use multiple struts, as shown in FIG. 14B. In addition, the use of multiple longitudinal and cross-braced struts may also be applied to impacted embodiments, with or without central spacers, as shown in FIGS. 15A-15C. The connecting struts may have an orientation that is wider in an anterior-to-posterior dimension than the superior-to-inferior dimension to keep the struts further from the end plate of the vertebrae, thereby allowing for an easier determination of fusion through x-ray analysis. The strut embodiments may also help to hold cancellous bone, which may be packed between and over the struts prior to insertion.

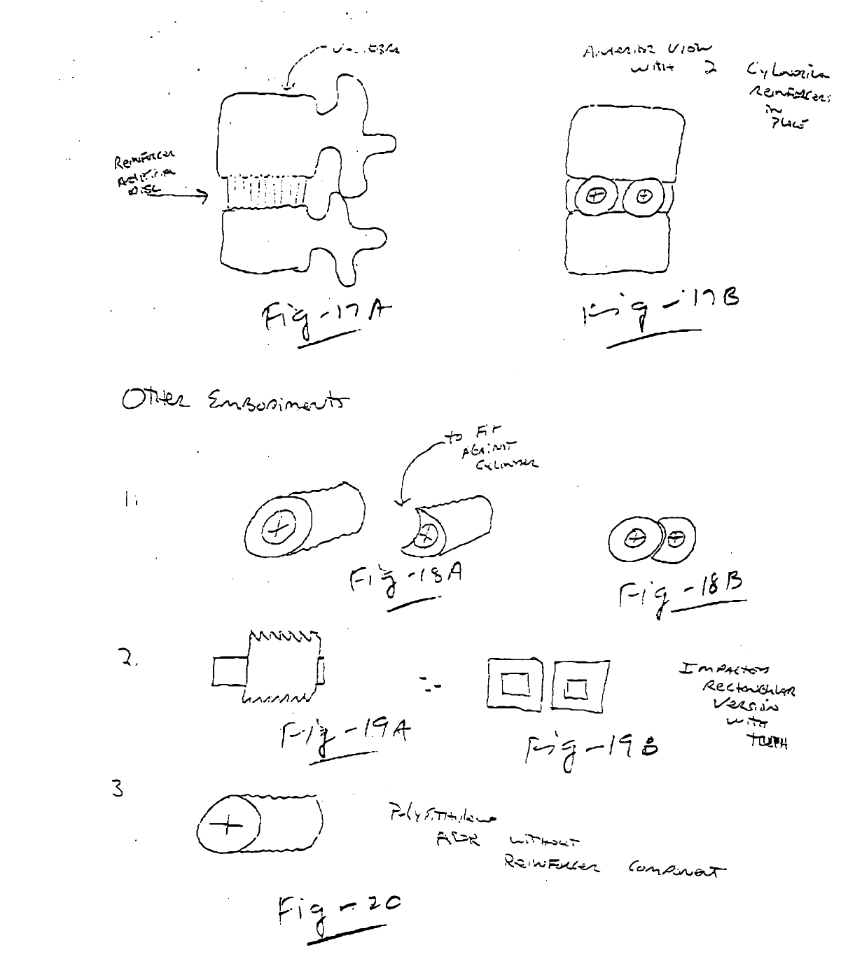

The devices described herein may also include a mid section composed of rubber, elastomers, polymers or other compressible/resilient materials. Such devices would allow a cylindrical or other shaped disc replacement to be screwed or impacted to consume at least a portion of the disc space. FIG. 16A is a side view of a device according to the invention wherein a compressible/resilient material surrounds a central reinforcer. FIG. 16B is an end-view of the device of FIG. 16A. FIG. 17A is a side-view drawing of the reinforcer of the type shown in FIGS. 16A and 16B, disposed between adjacent vertebra.

FIG. 17B is an anterior view showing the two cylindrical reinforcers in place. FIG. 18A is a drawing of an alternative embodiment of the invention from a perspective viewing how one or more covered reinforcers according to the invention may be shaped to fit against adjacent reinforcers. FIG. 18B shows the two devices of FIG. 18A cooperating with one another.

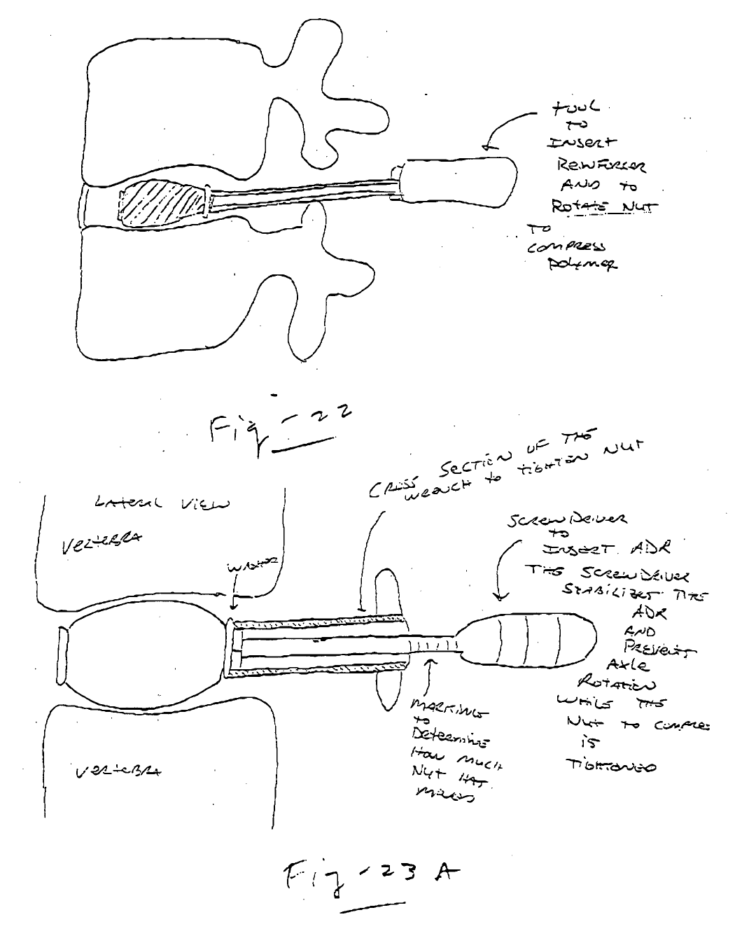

FIG. 19A illustrates yet a further embodiment of the invention, including an impacted rectangular shape with surface teeth or roughenings to prevent migration of the device. FIG. 19B is an end-view of two devices of the type shown in FIG. 19A. Alternatively, the artificial disc device could be made of a stiffer material such as polyethylene, as shown in FIG. 20. Reinforcers would not be necessary with polyethylene-like devices. In all such cases, surface threads, teeth, or roughening may be used to help prevent migration of the disc replacement.

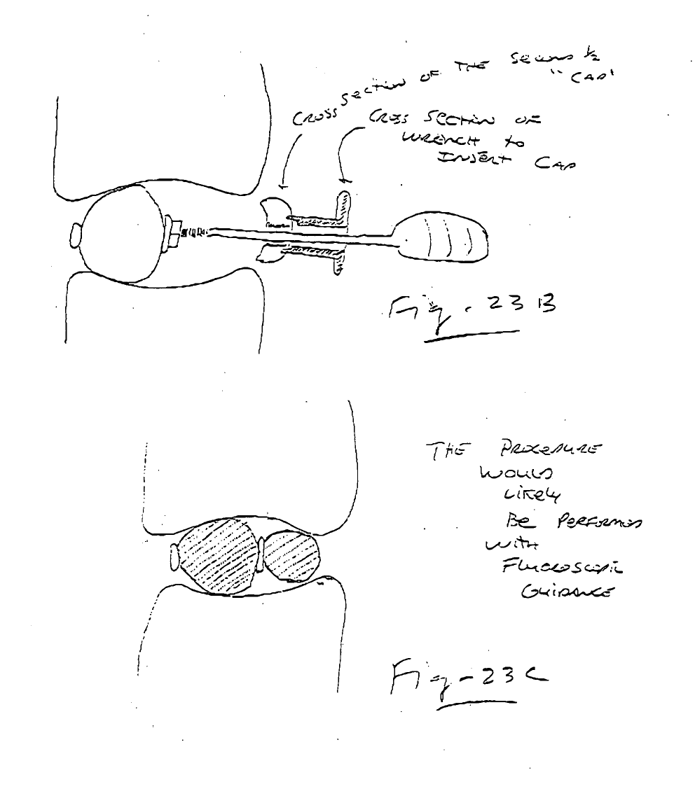

Depending upon the configuration, the reinforcers according to this invention may include a mechanical to compress the rubber or other material. Such “compression reinforcers” could also be used to increase the pressure or the height of the artificial disc device, as described in U.S. patent application Ser. No. 09/415,382. FIG. 21A illustrates this embodiment of the invention in a pre-compressed/expanded condition. FIG. 21B shows the device of FIG. 21A, with a nut moved along a reinforcing shaft, thereby causing the rubber, elastomeric or other material to bulge. FIG. 21C is a cross-section of the devices of FIGS. 21A and 21B. FIG. 21D is an end-view of the devices of FIGS. 21A-21C.

Broadly, the method of installing devices of this type would include the following steps:

1. Cutting a. hole or holes in the annulus fibrous;

2. Distracting the disc space by impacting a bullet or wedge-shape distracting tool into the disc space;

3. Drilling and tapping a hole for cylindrical shaped reinforcers while maintaining the disc space distraction; and

4. Screwing or tapping the disc replacement device into position.

FIG. 22 is a lateral drawing used to show the way in which instrumentation may be used to expand an enforcer covered with rubber, elastomeric/polymeric or other compressible/resilient material according to the invention. FIG. 23A shows how, after forming a hole in the annulus fibrosis and removing perhaps a portion of the nucleus pulposis, a wrench is used to tighten onto a rod, thereby expanding the reinforcer in place. Markings may be used to determine how much the nut has moved. A proximal handle stabilizes the disc replacement device, while preventing axial rotation as the nut is compressed using the wrench.

The invention is not limited to a single such device being could to a common axis. For example, as shown in FIG. 23B, a second expandable member or cap may be added to the device. FIG. 23C shows the final appearance of the structure, having performed the steps of FIGS. 23A and 23B. Such procedure would likely be performed with fluoroscopic guidance.

The preferred embodiment would be placed through an anterior approach to the cervical or lumbar spine. The devices could also be placed through a posterior approach to the spine. Posterior motion restriction devices of the type disclosed in co-pending U.S. application Ser. No. ______ may be used in conjunction with the device. The methods disclosed in U.S. Pat. Nos. 6,224,593 and 6,270,498 to insert fusion cages may alternatively be used.

I claim:

1. A reinforced plug for use within an intradiscal space between adjacent vertebra, the plug comprising:
a body having opposing ends dimensioned to fit within the intradiscal space; and
a section of rubber, polymeric, elastomeric or other compressible or resilient material disposed between the opposing ends.

2. The reinforced plug of claim 1, further including a pair of end plates disposed on either end of the section of rubber, polymeric, elastomeric or other compressible or resilient material.

3. The reinforced plug of claim 2, further including a link member interconnecting the end plates.

4. The reinforced plug of claim 3, wherein at least one of the end plates is positionable along the link member causing section of rubber, polymeric, elastomeric or other compressible or resilient material to bulge outwardly when the end plates are brought toward one another.

5. The reinforced plug of claim 2, wherein the end plates are at least partially metallic.

6. The reinforced plug of claim 2, wherein:
one of the end plates includes a threaded bore; and
the link member is threaded to receive the threaded bore of that end plate.

7. The reinforced bone plug of claim 2, wherein the mid section in combination with the end plates assumes a generally cylindrical, threaded configuration permitting a screw-in installation.

8. The reinforced bone plug of claim 2, wherein the mid section in combination with the end plates assumes a configuration suitable to an impacted installation.
